(19) United States
(12) Patent Application Publication
(10) Pub. No.: US 2015/0011941 A1
(43) Pub. Date: Jan. 8, 2015

(54) PHARMACEUTICAL PREPARATION-INJECTING INSTRUMENT

(71) Applicant: Masanori SAEKI, Tamano-shi, Okayama (JP)

(72) Inventor: Masanori Saeki, Tamano-shi (JP)

(21) Appl. No.: 14/380,616

(22) PCT Filed: Feb. 22, 2013
(86) PCT No.: PCT/JP2013/054430

§ 371 Date: Aug. 22, 2014

(30) Foreign Application Priority Data
Feb. 24, 2012 (JP) 2012-038396

(51) Int. Cl. A61B 17/34

(52) U.S. Cl. CPC A61B 17/3417; A61B 17/3472; A61B 2017/3454
USPC 604/164.06; 604/164.01

(57) ABSTRACT

A pharmaceutical preparation injecting instrument includes an outer needle and an inner needle. The outer needle has a straight tube shape having a distal side opening and a proximal side opening, and has a reference puncturing part on a distal side. The inner needle is configured to be removably inserted in the outer needle, and has a periphery puncturing part on a distal side. The inner needle is configured to allow the periphery puncturing part to appear from, or disappear into, the distal side opening in accordance with an advancing or withdrawing operation on the base side. When the inner needle is removed from the outer needle, the instrument is capable of injecting, from the distal side opening, a pharmaceutical preparation supplied from the proximal side opening. The outer needle guides the inner needle to project the periphery puncturing part to a side opposite the reference puncturing part.

### TECHNICAL FIELD

The present invention relates to a pharmaceutical preparation injecting instrument for injecting a pharmaceutical preparation into a patient's body and, more specifically, relates to a pharmaceutical preparation injecting instrument capable of enhancing the therapeutic effect of a pharmaceutical preparation by stimulating the interior of the body.

### BACKGROUND ART

As a conventional pharmaceutical preparation injecting instrument, for example, the drug injecting catheter disclosed in Patent Literature 1 is known. In this drug injecting catheter, an inner tube having an injection needle at the tip is slidably inserted in an outer tube. The inner tube is provided with a drug injecting lumen, and the injection needle of the inner tube is caused to project from the outer tube to puncture a lesion, thus making it possible to inject a drug.

### CITATION LIST

### Patent Literature

[PTL 1] JP 2001-104487A

### SUMMARY OF INVENTION

### Technical Problem

While the above-described conventional drug injecting catheter is capable of localized puncturing and drug injection, stimulation given to the interior of the body is not sufficient, and, therefore, there is room for improvement in enhancement of therapeutic effect.

Accordingly, an object of the present invention is to provide a pharmaceutical preparation injecting instrument capable of easily enhancing the therapeutic effect of a pharmaceutical preparation.

### Solution to Problem

The above object of the present invention is achieved by a pharmaceutical preparation injecting instrument comprising an outer needle that is formed in a straight tube shape having a distal side opening and a proximal side opening and has a reference puncturing part on a distal side and an inner needle that is removably inserted in the outer needle and has a periphery puncturing part on a distal side, wherein the inner needle is configured to be capable of allowing the periphery puncturing part to appear from or disappear into the distal side opening in accordance with an advancing or withdrawing operation on a base side, in a state in which the inner needle is removed from the outer needle, the pharmaceutical preparation injecting instrument is capable of injecting from the distal side opening a pharmaceutical preparation supplied from the proximal side opening, and the outer needle guides the inner needle such that the periphery puncturing part projects to a side opposite to the reference puncturing part across a plane including an axial line.

In this pharmaceutical preparation injecting instrument, it is preferable that the reference puncturing part has a puncturing direction that is parallel to the axial line of the outer needle, and it is preferable that the periphery puncturing part moves away from the axial line as it advances from the distal side opening.

It is preferable that the inner needle includes a holding part for regulating the length of projection from the distal side opening. In this configuration, it is preferable that the holding part is composed of a tapered part having a diameter that gradually decreases from the proximal side toward the distal side, and it is preferable that the holding part is held by the circumference of the distal side opening.

It is preferable that the distal side opening is formed in an inclined surface formed on the distal side of the outer needle so as to be off-center relative to the center of the outer needle, and it is preferable that the tip of the reference puncturing part is located on the side opposite to the center of the distal side opening across the center of the outer needle as viewed from the front.

It is preferable that the outer needle includes on the base side a marked part that allows the position of the periphery puncturing part relative to the reference puncturing part to be recognized.

In each pharmaceutical preparation injecting instrument described above, it is preferable that the outer needle is capable of insertion into a gap in a joint at which bones are joined to each other, and it is preferable that in a state in which the cartilage of one bone is punctured by the reference puncturing part, the periphery puncturing part is capable of puncturing the cartilage of another bone.

### Advantageous Effects of Invention

According to the present invention, a pharmaceutical preparation injecting instrument can be provided that is capable of easily enhancing the therapeutic effect of a pharmaceutical preparation.

### BRIEF DESCRIPTION OF DRAWINGS

FIG. 1 is a plan view of a pharmaceutical preparation injecting instrument according to one embodiment of the present invention.

FIG. 2 is an enlarged view of relevant parts of the outer needle of the pharmaceutical preparation injecting instrument shown in FIG. 1.

FIG. 3 shows cross-sectional views of relevant parts each showing a state during use of the pharmaceutical preparation injecting instrument shown in FIG. 1.

FIG. 4 is a schematic view for explaining an example of a treatment method in which the pharmaceutical preparation injecting instrument shown in FIG. 1 is used.

FIG. 5 shows cross-sectional views of relevant parts of a pharmaceutical preparation injecting instrument according to another embodiment of the present invention.

### DESCRIPTION OF EMBODIMENTS

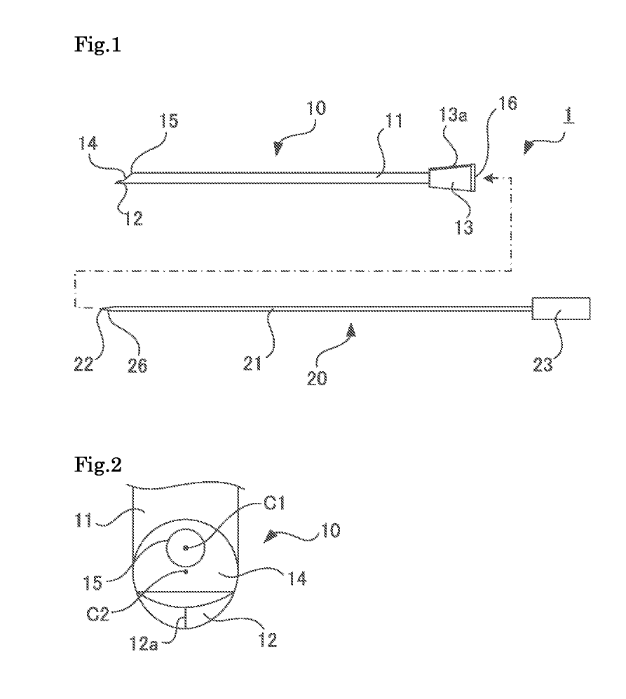

Below, embodiments of the present invention will now be described with reference to the attached drawings. FIG. 1 is a plan view of a pharmaceutical preparation injecting instrument according to one embodiment of the present invention. A pharmaceutical preparation injecting instrument 1 includes an outer needle 10 and an inner needle 20 that is removably inserted in the outer needle 10. FIG. 1 shows the outer needle 10 and the inner needle 20 separately.

The outer needle 10 includes a flexible main body 11 composed of, for example, metal such as stainless steel or synthetic resin such as polyethylene, and a sharp-edged reference puncturing part 12 is provided on the distal side of the main body 11. The main body 11 is a straight tube having a distal side opening 15 and a proximal side opening 16 on the distal side and the proximal side, respectively. A cylindrical attachment part 13 is provided on the proximal side of the main body 11. The tip part of a syringe (not shown) for supplying a pharmaceutical preparation or the like can be attached to the attachment part 13 by, for example, screw-fitting. The outer needle 10 configured in this way is capable of discharging from the distal side opening 15 a liquid pharmaceutical preparation, rinse water, or the like supplied from the proximal side opening 16, thus enabling injection of a pharmaceutical preparation, washing of the outer needle 10, etc.

FIG. 2 is a perspective front view of the distal side of the outer needle 10 shown in FIG. 1 as viewed obliquely from above. As shown in FIGS. 1 and 2, the tip surface of the outer needle 10 is an inclined surface 14, and the distal side opening 15 is formed in this inclined surface 14. A center C1 of the distal side opening 15 is off-center relative to a center C2 through which the axial line of the outer needle 10 passes, and the distal side opening 15 opens above the center C2 of the inclined surface 14. A tip 12a of the reference puncturing part 12 is located on the side opposite to the center C1 of the distal side opening 15 across the center C2 of the outer needle 10, and is provided so as to project from the lower part of the inclined surface 14. Instead of configuring the reference puncturing part 12 to project from the inclined surface 14, the lower part itself of the inclined surface 14 may be configured to be the reference puncturing part 12. A marked part 13a is provided on the upper part of the attachment part 13 of the outer needle 10 such that the positions in the circumferential direction of the reference puncturing part 12 and the distal side opening 15 can be recognized on the base side of the outer needle 10.

As shown in FIG. 1, the inner needle 20 includes a flexible main body 21 composed of a material such as metal or synthetic resin, as with the main body 11 of the outer needle 10. The main body 21 is a solid bar. A tapered part 26 having a diameter that gradually decreases from the proximal side toward the distal side is formed on the distal side of the main body 21, and a sharp-edged periphery puncturing part 22 is formed at the tip of the tapered part 26. A grip 23 is provided on the base side of the main body 21. Regarding the inner needle 20, inserting the distal side into the proximal side opening 16 of the outer needle 10 and performing advancing or withdrawing operations by grasping the grip 23 allow the periphery puncturing part 22 to appear from or disappear into the distal side opening 15.

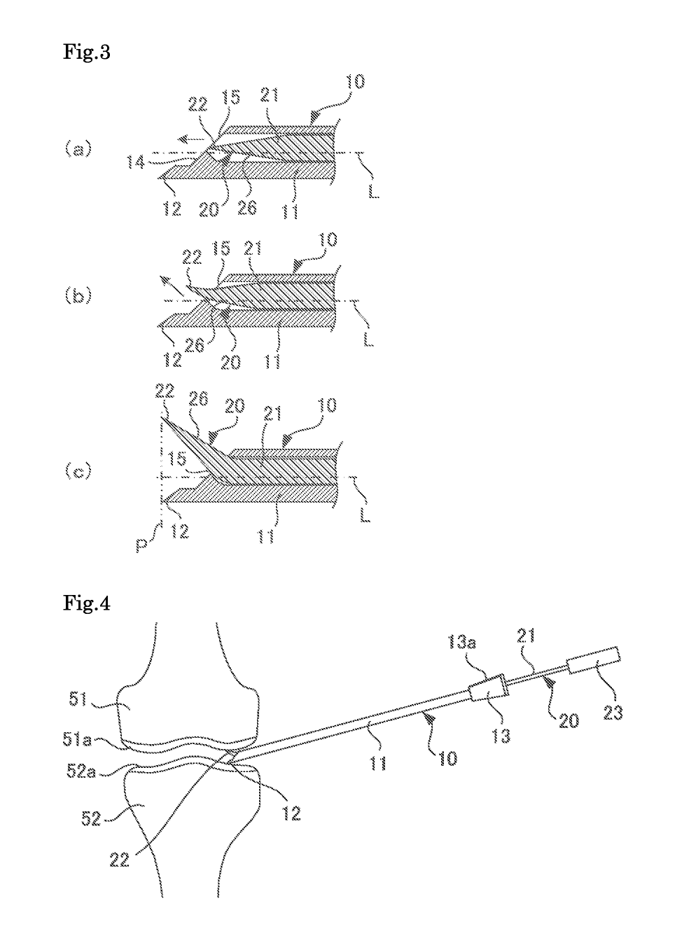

With the pharmaceutical preparation injecting instrument 1 having the above configuration, when the inner needle 20 is inserted into the outer needle 10, and the inner needle 20 is advanced so as to cause the periphery puncturing part 22 to project from the distal side opening 15 as indicated by the arrow shown in FIG. 3(a), the lower part of the tapered part 26 comes into contact with the circumference of the distal side opening 15 as shown in FIG. 3(b). When the inner needle 20 is further advanced, the periphery puncturing part 22 changes its direction so as to move in the normal-line direction of the distal side opening 15 as indicated by the arrow in FIG. 3(b), and further projects from the distal side opening 15. And, as shown in FIG. 3(c), the entire outer circumference of the tapered part 26 is held by the distal side opening 15, and thus the inner needle 20 is held by the outer needle 10.

With the pharmaceutical preparation injecting instrument 1 of this embodiment, as shown in FIGS. 3(a) to (c), the inner needle 20 is guided through the interior of the outer needle 10 such that the periphery puncturing part 22 projects to the side opposite to the reference puncturing part 12 across the transverse plane including an axial line L of the outer needle 10 (the plane perpendicular to the paper surface in FIG. 3), and it is therefore possible, in addition to puncturing by the reference puncturing part 12, to puncture by the periphery puncturing part 22 a place away from the place of puncture by the reference puncturing part 12. Accordingly, it is possible, before injecting a pharmaceutical preparation, to easily stimulate the periphery of a site where the pharmaceutical preparation is going to be injected, and thus the therapeutic effect by the pharmaceutical preparation can be enhanced.

FIG. 4 is a drawing for explaining a treatment method for the knee joint of a human body as an example of a treatment method in which the pharmaceutical preparation injecting instrument 1 of this embodiment is used. As shown in FIG. 4, the tip part of the outer needle 10 is inserted into the gap between a femur 51 and a tibia 52 that constitute a knee joint 50, and the inner needle 20 is caused to advance, with a cartilage 52a of the tibia 52 (or a cartilage 51a of the femur 51) being in a punctured state by the reference puncturing part 12, to puncture the cartilage 51a of the femur 51 (or the cartilage 52a of the tibia 52) by the periphery puncturing part 22. Thereby, the cartilages 51a and 52a of the femur 51 and the tibia 52, respectively, are stimulated. With the reference puncturing part 12 being in a puncturing state, rotating the outer needle 10 makes it possible to promptly and easily puncture another site in the periphery of the reference puncturing part 12 by the periphery puncturing part 22. In this way, puncturing multiple places of the cartilages 51a and 52a of the femur 51 and the tibia 52, respectively, by the reference puncturing part 12 and also puncturing by the periphery puncturing part 22 multiple places in the periphery of the places punctured by the reference puncturing part 12 make it possible to effectively stimulate the interior of the body. Thereafter, the inner needle 20 is pulled out from the outer needle 10, a syringe or the like accommodating a pharmaceutical preparation is attached to the attachment part 13, and the pharmaceutical preparation is supplied to the gap in the knee joint 50 from the distal side opening 15, thus making it possible to enhance the therapeutic effect by the synergy between the pharmaceutical preparation and the inner-body stimulation.

In the pharmaceutical preparation injecting instrument 1 of this embodiment, as shown in FIGS. 3(a) to (c), the direction of puncture of the reference puncturing part 12 is parallel to the axial line L of the outer needle 10, while the periphery puncturing part 22 moves away from the axial line L as it advances from the distal side opening 15. This configuration makes it possible to easily puncture by the periphery puncturing part 22 a place away from the place of puncture by the reference puncturing part 12 and makes it possible to expand the region where the periphery puncturing part 22 can puncture, and it is thus possible to more efficiently give stimulation to the interior of the body.

With the pharmaceutical preparation injecting instrument 1 of this embodiment, the tapered part 26 of the inner needle 20 is held by the circumferential part of the distal side opening 15 and thus the length of projection is regulated. Therefore, when advancement and withdrawal of the periphery puncturing part 22 are repetitively performed while rotating the outer needle 10, it is possible to suppress excessive advancement of the periphery puncturing part 22 and give appropriate stimulation, thus enabling puncturing to be effectively and safely performed. The length of projection of the inner needle 20 from the distal side opening 15 may be suitably set according to the application of the pharmaceutical preparation injecting instrument 1. For example, when it is used for treatment of the knee joint as shown in FIG. 4, the length of projection may be set such that, with one of the cartilages 51a and 52a of the femur 51 and the tibia 52 being in a punctured state by the reference puncturing part 12, the other can be punctured by the periphery puncturing part 22. In this embodiment, as shown in FIG. 3(c), the length of projection of the inner needle 20 is set such that the tip of the reference puncturing part 12 and the tip of the periphery puncturing part 22 in its furthest projecting position are on the same plane P that is perpendicular to the axial line L of the outer needle 10.

Since the pharmaceutical preparation injecting instrument 1 of this embodiment is provided with the marked part 13a on the proximal side of the outer needle 10, it is possible to easily know the position of the periphery puncturing part 22 relative to the reference puncturing part 12 by the marked part 13a also in a state in which the distal side of the outer needle 10 is inserted in the body. Therefore, the desired puncturing operation can be efficiently performed.

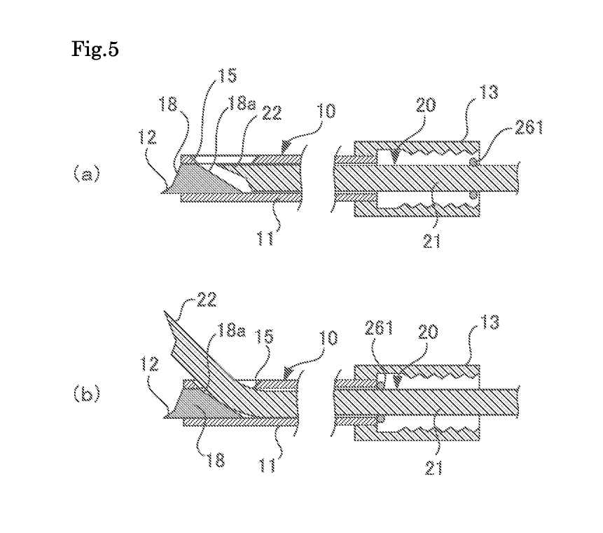

In this embodiment, the distal side opening 15 is formed in the inclined surface 14 on the distal side of the outer needle 10, but it is also possible to form the distal side opening 15 in the side wall of the outer-needle main body 11 as shown in FIG. 5(a). In this case, inserting a guiding member 18 into the distal side of the main body 11 such that a guiding surface 18a of the guiding member 18 faces the distal side of the inner needle 20 causes the distal side of the inner needle 20 to be guided along the guiding surface 18a to the distal side opening 15 as shown in FIG. 5(b), thus enabling smooth advancement of the inner needle 20. Furthermore, in this configuration, it is possible to provide the reference puncturing part 12 on the guiding member 18 instead of providing it on the main body 11 of the outer needle 10. In FIG. 5, the same components as in FIGS. 1 to 3 are given the same reference numbers.

Moreover, in this embodiment, the tapered part 26 is formed on the distal side of the inner needle 20, and the circumference of the tapered part 26 is held by the circumference of the distal side opening 15, thus regulating the length of projection of the inner needle 20 from the distal side opening 15. However, such a holding part for regulating the length of projection does not necessarily need to be the tapered part 26. For example, as shown in FIGS. 5(a) and (b), fixing a ring-shaped member 261 to the proximal side of the inner needle 20 to configure the ring-shaped member 261 to be held by the proximal-end face of the main body 11 of the outer needle 10 due to the advancement of the inner needle 20 makes it possible to set the length of projection of the inner needle 20 to the desired value.

The pharmaceutical preparation injecting instrument of the present invention is preferably usable for treatment of an interosseous joint as will be described below, but is not limited to this application, and it is also usable for treatment of mesoderm-derived organs other than the interosseous joint (such as dermis, muscle, bone/cartilage, gonad, spermatic duct, oviduct, womb, pericardial membrane, pleura, peritoneum, endocardium, and vascular intima).

As pharmaceutical preparations for injection into an interosseous joint such as a knee joint, it is possible to use known pharmaceutical preparations used for conventional joint treatment, such as hyaluronic acid and steroid, but it is preferable to use cell preparations containing adipocytes.

The inventors have found that injection of adipocytes into a patient suffering from a joint pain promptly relieves the symptoms and that the effect lasts for a long period of time. Furthermore, it was found that injection of adipocytes and adipose-derived stem cells in combination brings about, in addition to the aforementioned relief of joint pain, efficient reproduction of bone tissue and cartilage tissue in the joint. Furthermore, it was found that, before injecting these, giving appropriate stimulation to the vicinity of the site of injection using the pharmaceutical preparation injecting instrument of the present invention further enhances the therapeutic effect. That is, due to the use of the pharmaceutical preparation injecting instrument of the present invention, the present invention can provide an animal or human-body treatment method having the following aspects.

Item 1. A treatment method comprising giving stimulation to a diseased site by puncturing a target site using the pharmaceutical preparation injecting instrument of the present invention and then injecting a cell preparation containing adipocytes into the diseased site.
Item 2. The treatment method according to item 1, wherein the cell preparation further contains mesodermal stem cells.
Item 3. The treatment method according to item 2, wherein the mesodermal stem cells are adipose-derived stem cells.
Item 4. The treatment method according to item 2 or 3, wherein 1 to 10 mesodermal stem cells are contained per adipocyte.
Item 5. The treatment method according to any of items 1 to 4, wherein the adipocytes are γ-irradiated cells.
Item 6. The treatment method according to any of items 1 to 5, wherein the diseased site is an interosseous joint.
Item 7. The treatment method according to item 6, wherein the disease of the interosseous joint is one selected from the group consisting of spine diseases, joint diseases, autoimmune diseases, and osteonecroses.

According to the treatment method of the present invention, it is possible to improve sliding of a joint or the like by a fat cell, an ECM (extracellular matrix) existing around a fat cell or the like, thereby enabling early alleviation of arthralgia, and an excellent effect in alleviation of pain caused by a disease of an interosseous joint can be obtained. In addition, for example, by injecting the cell preparation to an intervertebral disk or the like, the cell preparation serves as a cushion and exhibits an effect of making motion of a vertebral body good (improvement). Furthermore, the effect of alleviating arthralgia by the cell preparation lasts for a long time.

In addition, according to the cell preparation including a fat cell and a mesodermal stem cell, pain caused by a disease of an interosseous joint is alleviated by the fat cell, during which time regeneration of a tissue in the joint such as a bone tissue, a cartilage tissue or a soft tissue by the mesodermal stem cell is enhanced. In addition, by using a combination of a fat cell and a mesodermal stem cell, not only the above-mentioned pain can be alleviated, but also take percentage of the mesodermal stem cell and efficiency of regeneration of the tissue can be still more enhanced, since the mesodermal stem cell is retained in the affected area by the action of the fat cell. Thus, the cell preparation including a fat cell and a mesodermal stem cell is further effective for treatment of a disease of an interosseous joint.

Conventionally, when a method of restoring a tissue by culturing a mesodermal stem cell such as an adipose stem cell is employed, there has been a possibility of contamination in the course of culturing and the like, and an infectious disease could have been caused after transplantation. However, since the fat cell and the mesodermal stem cell included in the cell preparation used in the treatment method of the present invention can be directly separated and concentrated from the sucked adipose or the like without undergoing a step of cell culturing, the fat cell and the mesodermal stem cell can be prepared without contacting with the open air. Therefore, there is no danger of an infectious disease or the like. In addition, since the cell preparation used in the treatment method of the present invention is generally prepared using a fat cell and a mesodermal stem cell collected from a patient himself/herself who requires treatment of a disease of an interosseous joint or restoration of a muscle, the cell preparation has no problem of immunorejection and is highly safe.

1. Cell Preparation

A fat cell is a cell which has functions of synthesizing, storing and releasing fat and which forms an adipose tissue. In addition, since a fat cell has been completely differentiated, the cell does not undergo cell division. The fat cell is classified into a white fat cell and a brown fat cell. Either of the cells may be used in the present invention, or a mixture of both the cells may be used. In the present invention, the origin of the fat cell is not specifically limited, and, for example, a fat cell of a mammal, preferably a fat cell of a primate, further preferably a human fat cell is used. In the present invention, as a fat cell, a fat cell alone may be used, or a mixture of an extracellular matrix (ECM) existing around a fat cell and a fat cell may be used. Specifically, in the present invention, a collected fat cell may be used as it is, or a fat cell prepared by removing the extracellular matrix from the collected fat cell may be used.

A fat cell is collected from an adipose tissue. A method for collecting an adipose tissue is not specifically limited, and appropriately selected from conventionally known methods, including, for example, liposuction, lipectomy, and defatting. Collection by liposuction is preferable, since liposuction is relatively easy and minimally invasive. A method for obtaining a fat cell from the collected fat is not specifically limited, and is preferably the method of 2. Method for preparing a cell preparation described below.

In addition, the cell preparation used in the treatment method of the present invention may further include a mesodermal stem cell. By including a mesodermal stem cell, regeneration of a tissue in the interosseous joint is enhanced, and the therapeutic effect by the cell preparation is still more increased.

The mesodermal stem cell used for the cell preparation used in the treatment method of the present invention mainly indicates an intermediate stem cell which is to differentiate to a cell belonging to a mesodermal tissue such as bone, cartilage, myocardium, and fat. The origin of the mesodermal stem cell is not specifically limited, and, for example, a mesodermal stem cell of a mammal, preferably a mesodermal stem cell of a primate, further preferably a human mesodermal stem cell is used.

As an example of a mesodermal stem cell, an adipose stem cell, a bone marrow stem cell, a hematopoietic stem cell and the like are specifically mentioned, and these stem cells can be obtained from each tissue depending on the kind of the cell in accordance with a conventionally known method.

In the present invention, as an example of a mesodermal stem cell, an adipose stem cell is preferably mentioned. An adipose stem cell can be collected from an adipose tissue. The adipose tissue used as the source of the adipose stem cell can also be collected by any of the methods such as liposuction, lipectomy and defatting as with the above-mentioned fat cell, and the method is preferably liposuction from the viewpoint of preventing outflow or damage of the adipose stem cell and the viewpoint of safer collection. In addition, a preferable example of a method for obtaining an adipose stem cell from the collected fat is the method described in 2. Method for preparing a cell preparation described below.

When the cell preparation used in the treatment method of the present invention includes a mesodermal stem cell, it is preferable that the mesodermal stem cell in a state of a mixture with a fat cell be used. When the cell preparation used in the treatment method of the present invention includes a mesodermal stem cell, the content of the mesodermal stem cell in the cell preparation is not specifically limited so long as a therapeutic effect on a disease of an interosseous joint can be obtained. While it can be thought that the higher the content of the mesodermal stem cell is, the more excellent therapeutic effect can be obtained, the content is, for example, at least 1, preferably 5 or more, more preferably 1 to 10, further preferably 5 to 10 mesodermal stem cells per one fat cell. In addition, in a case of a cell preparation prepared by the method (c) described below including the steps of (1c) and (2c), the number of the mesodermal stem cells included in 1 mL of the cell preparation is generally 100 thousand or more, preferably 150 thousand or more, further preferably 150 to 200 thousand.

In the cell preparation used in the treatment method of the present invention, by using an autologous fat cell (and an autologous mesodermal stem cell) for the purpose of autologous transplantation, immunorejection can be avoided in addition to enablement of early improvement of the symptom. However, preparation of the cell preparation used in the treatment method of the present invention using a fat cell (and a mesodermal stem cell) derived from another person is not limited by the above. When using a cell derived from another person, it is preferable to carry out treatment for removing antigenicity such as, for example, gamma irradiation for the fat cell (including the extracellular matrix (ECM) existing around the fat cell) collected from another person before transplantation. While the mesodermal stem cell is a cell generally having no immunogenicity, a mesodermal stem cell which has been subjected to the above-mentioned treatment may also be used, if needed.

As a preferable example of the cell preparation used in the treatment method of the present invention, a cell preparation including a fat cell and an adipose stem cell can be mentioned. Generally, an adipose tissue includes a fat cell and an adipose stem cell. Thus, as the cell preparation including a fat cell and an adipose stem cell, a collected adipose tissue may be used as it is, or a cell preparation prepared by removing impurities (for example, an anesthetic solution injected upon collection of fat (so-called tumescent fluid), an aged fat cell, blood, and tissue fluid) from the collected adipose tissue, a cell preparation prepared by removing the impurities and a part of a fat cell from the collected adipose tissue, and the like may be used. Preferable examples of a method for preparing the cell preparation include methods (a) to (c) described below.

In addition, a conventionally known pharmaceutically acceptable carrier, excipient, antiphlogistic, analgesic, immunosuppressive or the like can be added to the cell preparation used in the treatment method of the present invention, if needed. In addition, collagen, a fibroblast, a growth factor, a proliferator, a cytokine or the like may be added thereto so long as the effect of the present invention is not negated.

The dose of the cell preparation used in the treatment method of the present invention is not specifically limited so long as the dose can give a therapeutic effect on the disease of an interosseous joint, and can be appropriately set depending on the size of the part of administration, the site of the disease, the degree of the disease, and sex, physique and the like of the patient. For example, the dose is 100 thousand to 4 million cells, preferably 200 thousand to 2 million cells, further preferably 200 thousand to 1 million cells as converted into the number of the fat cells. For example, when a cell preparation obtained by the method (c) described below including the steps of (1c) and (2c) is used, the dose is 0.5 to 20 mL, preferably 1 to 10 mL, further preferably 1 to 5 mL. In addition, for example, regarding the knee joint, 1 to 20 mL, preferably 1 to 10 mL, further preferably 5 to 10 mL of the cell preparation can be administered. In addition, regarding the intervertebral disk, 0.5 to 20 mL, preferably 1 to 15 mL, further preferably 1 to 2 mL of the cell preparation can be administered.

The cell preparation is injected after puncturing the vicinity of the diseased interosseous joint (for example, joint space and intervertebral disk (intervertebral disc)) by the reference puncturing part and also puncturing by the periphery puncturing part multiple places in the periphery of the places punctured by the reference puncturing part using the pharmaceutical preparation injecting instrument of the present invention. Thus, excellent therapeutic effect can be obtained by giving stimulation to a diseased site or its neighborhood by puncturing a target site and then injecting a cell preparation. The target site to be punctured is not limited to the diseased site or its neighborhood. The other part may be punctured if the diseased site or its neighborhood can be stimulated.

The cell preparation used in the treatment method of the present invention exhibits an excellent therapeutic effect on a disease of an interosseous joint. The interosseous joint indicates a connection part of bones, and encompasses a cartilaginous joint connected by a cartilage or a combination of a cartilage and a fibrous connective tissue, a synovial joint connected by a cartilage and a synovial membrane covering a joint capsule, and a fibrous joint connected by a fibrous connective tissue. In the present invention, the interosseous joint especially indicates a part having mobility connected by a cartilaginous joint and a synovial joint. As an example of the interosseous joint, a vertebral joint, a knee joint, a foot joint, a toe joint, a hip joint, a hand joint, an elbow joint, a shoulder joint, a finger joint, an acromioclavicular joint, a sacroiliac joint and the like can be specifically mentioned. In addition, for example, in a case of a knee joint, soft tissues such as a ligament and a meniscus are encompassed by the above-mentioned interosseous joint, and in a case of a vertebral joint, a disk (intervertebral disk) and the like are encompassed by the above-mentioned interosseous joint.

In addition, examples of the disease of an interosseous joint include hernia (prolapse), deformation, degeneration, and inflammation, and specifically include spinal diseases such as intervertebral disk displacement and spondylosis deformans; joint diseases such as osteoarthritis (including hip osteoarthritis and knee osteoarthritis) and chronic arthrosis; autoimmune diseases such as rheumatoid arthritis; and osteonecrosis such as idiopathic osteonecrosis of knee joint and osteonecrosis of femoral head. Preferably, intervertebral disk displacement, osteoarthritis, chronic arthrosis, idiopathic osteonecrosis of knee joint and osteonecrosis of femoral head can be mentioned.

When the cell preparation is administered to patients having these diseases, the cell preparation may be administered after carrying out a treatment by a known method, if needed. For example, in a case of intervertebral disk displacement, the cell preparation can be administered (injected), after removing the prolapsed intervertebral disk by a conventionally known method. By the action of the fat cell included in the cell preparation, pain caused by the disease of an interosseous joint (arthralgia) is alleviated, and, furthermore, when a mesodermal stem cell is included, a bone tissue, a cartilage tissue, a soft tissue or the like in the interosseous joint is regenerated by the action of the cell.

2. Method for Preparing a Cell Preparation

In the present invention, a fat cell can be collected, and, if needed, separated and concentrated to prepare the cell preparation. In addition, when preparing a cell preparation including a fat cell and a mesodermal stem cell, a fat cell collected as a mixture of these cells may be used, or each cell may be individually collected, and thereafter mixed with the other cell to give the cell preparation of the present invention. For example, when the mesodermal stem cell is an adipose stem cell, since the adipose stem cell can be simultaneously prepared from the same tissue as the fat cell and easily prepared, it is preferable to obtain a mixture of a fat cell and an adipose stem cell from an adipose tissue to give the cell preparation of the present invention. In addition, more easily, an adipose tissue including these cells may be used.

When preparing the cell preparation of the present invention from an adipose tissue, methods (a) to (c) described below can be mentioned as preferable methods. However, these methods do not limit preparation of the cell preparation of the present invention from a tissue other than fat, and the cell preparation of the present invention can also be prepared by appropriately setting conditions, considering the kind or properties of the cell according to the method described below, even when the cell preparation of the present invention is prepared from a tissue other than an adipose tissue (for example, blood, bone marrow aspirate, or muscle). Each of the methods (a) to (c) will be described in detail herein below.

Method (a)

The method (a) for preparing the cell preparation used in the treatment method of the present invention includes a step (1a) of removing a liquid fraction from the collected fat to give a cellular fraction.

Here, the collected fat indicates a mixture of a cellular fraction and a liquid fraction derived from an adipose tissue that is collected from an adipose tissue by liposuction, lipectomy, defatting or the like. The cellular fraction includes a fat cell, an adipose stem cell and impurities (hemocytes, fatal/vital cells, senescent cells and the like). In addition, the liquid fraction includes a tumescent fluid, a cellular tissue fluid or the like.

Separation of the liquid fraction from the cellular fraction can be easily carried out by precipitating the cellular fraction by leaving the collected fat standing still. Examples of the method for removing the liquid fraction include, but not specifically limited to, decantation and suction. In addition, centrifugation, filtration or the like can be carried out, if needed.

Conditions for centrifugation and filtration are not specifically limited so long as the fat cell and the adipose stem cell are not damaged and the impurities can be removed. Examples of the conditions include the conditions of at 700 to 2,500×g for 1 to 15 minutes, preferably the conditions of at 2,000 to 2,200×g for 5 to 10 minutes.

In addition, when filtration is carried out, for example, blood, tissue fluid, anesthetic administered to the part from which the fat has been collected (a tumescent fluid) and the like are removed by letting the collected fat through a mesh filter with pores having a size of 10 to 300 μm, preferably 10 to 150 μm, more preferably 10 to 100 μm, further preferably 15 to 20 μm to give a mixture of a fat cell and an adipose stem cell on the filter. In addition, a container equipped with such a mesh filter may be charged with the collected fat containing impurities, and the fat may be subjected to centrifugation.

Thus, an adipose tissue including a fat cell (including an extracellular matrix) and an adipose stem cell from which a liquid fraction has been removed can be obtained, and the adipose tissue can be used as the cell preparation.

Method (b)

The method (b) for preparing the cell preparation used in the treatment method of the present invention is characterized in that the method includes the following steps:
(1b) removing a liquid fraction from the collected fat to give a cellular fraction; and
(2b) separating a fat cell and an adipose stem cell from at least a part of the cellular fraction obtained in step (1b).

Here, step (1b) is as described in the above-mentioned step (1a). In step (2b), when a cell preparation including a fat cell is prepared, the whole of the cellular fraction obtained in step (1b) can be subjected to the treatment of step (2b) and only the separated fat cell can be used. In addition, when a cell preparation including a fat cell and an adipose stem cell is prepared, a cell preparation including a fat cell and an adipose stem cell can be obtained by separating an adipose stem cell from at least a part of, preferably half of the amount of the cellular fraction obtained in step (1b) and further adding the adipose stem cell to the cellular fraction obtained in step (1b).

Here, examples of the method for separating an adipose stem cell include the method described in JP 2005-519883. More specifically, in order to facilitate separation of a fat cell and an adipose stem cell by degrading intercellular junction, a protease is added to the cellular fraction. Examples of the protease include collagenase, trypsin, and lipase. One kind of the protease selected therefrom may be used alone, or two or more kinds of the proteases may be used in combination.

The method for separating a fat cell and an adipose stem cell from the cellular fraction treated with an enzyme is not specifically limited, and can be appropriately selected from the conventionally known methods. For example, the method may be centrifugation, filtration or the like. Conditions for centrifugation are not specifically limited so long as the fat cell and the adipose stem cell can be separated without damaging the cells. Examples of the conditions include the conditions of at 100 to 500×g for 1 to 20 minutes, preferably the conditions of at 100 to 300×g for 5 to 10 minutes. By subjecting the cellular fraction to such centrifugation, an adipose stem cell can be obtained as a precipitate, and a fat cell is concentrated in the supernatant. In addition, when filtration is carried out, an adipose stem cell can be obtained on a filter by letting the cellular fraction or a suspension thereof through a mesh filter with pores having a size of 10 to 100 μm, preferably 10 to 50 μm, further preferably 15 to 20 μm, and a fat cell can be obtained in the filtrate.

The fat cell and the adipose stem cell treated with an enzyme is preferably subjected to washing treatment after separation, and washing can be carried out with phosphate buffered saline (PBS), saline, Ringer's solution, dextran or the like. The washing treatment may be repeated multiple times if needed, or the cells may be subjected to centrifugation if needed, upon collecting the cells after washing. The conditions for centrifugation can follow the conditions employed upon separating a fat cell and an adipose stem cell from the cellular fraction treated with an enzyme.

After washing, the fat cell separated in the present step (2b) can be used as the cell preparation. The cell preparation obtained using the fat cell obtained in the present method (b) exhibits an excellent effect in alleviation of pain caused by a disease of an interosseous joint.

Furthermore, the present method (b) may include a step (3b) of mixing the cellular fraction obtained in step (1b) with the adipose stem cell obtained in step (2b) after step (2b). In the present step (3b), a cell preparation including a fat cell and an adipose stem cell can be obtained by mixing the cellular fraction obtained in step (1b) with the adipose stem cell obtained in step (2b). The cell preparation thus prepared includes an adipose stem cell in a high concentration, and not only is effective for alleviation of pain caused by a disease of an interosseous joint, but also exhibits an excellent effect on regeneration of a tissue of an interosseous joint. In addition, such a cell preparation including a fat cell and an adipose stem cell exhibits an excellent effect of restoration of a muscle on a damaged muscle, and simultaneously exhibits an excellent effect on alleviation of pain caused by muscle damage.

Method (c)

The method (c) for preparing a cell preparation used in the treatment method of the present invention includes the following steps of:
(1c) removing a liquid fraction from the collected fat to give a cellular fraction; and
(2c) removing impurities from the cellular fraction obtained in step (1c) to give a concentrate of a fat cell and an adipose stem cell.

Here, step (1c) is as described in step (1a). In step (2c), the cellular fraction is concentrated by removing impurities therefrom. The method for removing impurities and the method for concentration are not specifically limited, and can be appropriately selected from the conventionally known methods. As an easy method, for example, by using the syringe equipped with a weight filter described in JP 2007-533396, impurities such as free oil released from the broken fat cell, fatal/vital cells and senescent cells are removed to give a concentrate of a healthy fat cell and an adipose stem cell. Such a syringe is commercially available, and a specific example thereof is a syringe manufactured for LIPOMAX-SC (manufactured by Medikan International Inc., Seoul, Korea).

More specifically, a syringe equipped with a filter therein is charged with the collected fat, and the fat is separated into a liquid fraction and a cellular fraction by centrifugation. Furthermore, free oil (drainage oil) is separated by the filter. The liquid fraction separated here is a liquid which has not been completely separated from the cellular fraction and mixed therewith in step (1c). That is to say, in the syringe, free oil, a concentrate of a fat cell and an adipose stem cell, and a liquid fraction are separated in the upper layer, the intermediate layer and the lower layer, respectively. By discarding the layers other than the intermediate layer, a concentrate of a healthy fat cell and an adipose stem cell can be obtained, and the concentrate can be used as the cell preparation. Since a healthy fat cell and an adipose stem cell are selected and concentrated, the cell preparation thus obtained hardly causes necrosis, calcification or the like after transplantation, and is still more useful in alleviation and treatment of symptoms of a disease of an interosseous joint.

Conditions for the centrifugation are not specifically limited so long as the fat cell and the adipose stem cell are not damaged and the impurities are separated from the fat cell and the adipose stem cell. Examples of the conditions include the conditions of at 700 to 2,500×g for 1 to 15 minutes, preferably the conditions of at 2,000 to 2,200×g for 5 to 10 minutes.

In addition, the pore size of the filter is not specifically limited so long as a fat cell and an adipose stem cell cannot pass through the pore and impurities can pass through the pore. The size is, for example, 10 to 300 μm, preferably 10 to 150 μm, more preferably 10 to 100 μm, further preferably 15 to 20 μm.

In addition, the present method (c) may include a step (3c′) of separating a fat cell and an adipose stem cell from at least a part of the concentrate obtained in step (2c) and further mixing the obtained adipose stem cell with the concentrate obtained in step (2c). The method for separating an adipose stem cell is as described in step (2b) of the above-mentioned method (b). In the present step (3c′), when an adipose stem cell is separated using a protease, generally about 700 thousand or more, preferably about 1.5 million or more, further preferably about 2 million or more of an adipose stem cell per 1 cc of the concentrate obtained in step (2c) can be obtained. Since a healthy fat cell is selected and concentrated, and moreover an adipose stem cell is included in a higher concentration, the cell preparation prepared via the present step (3c′) exhibits a still more excellent effect on treatment of a disease of an interosseous joint.

In addition, the fat cell separated in the present step (3c′) can be washed, and thereafter used as the cell preparation of the present invention. Since a healthy fat cell is selected and concentrated, the fat cell obtained here exhibits a still more excellent effect in alleviation of a disease of an interosseous joint or pain caused by muscle damage.

In addition, the present method (c) may include a step (3c″) of micronizing the fat cell in the obtained concentrate after step (2c), and separating the micronized fat cell and an adipose stem cell as a mixture. Micronization of the fat cell can be carried out by a method generally employed in the art, and is not specifically limited. For example, a fat cell is cut with a drill or the like if needed, and thereafter a mixture of the micronized fat cell and an adipose stem cell is separated. As the method for separation, the above-mentioned conditions for centrifugation can be employed. Upon carrying out the present step (3c″), after step (2c), the concentrate may be subjected to micronization and centrifugation of a fat cell while the concentrate is remaining in the syringe. Since free oil is separated from the mixture of the micronized fat cell and an adipose stem cell after centrifugation, the free oil can be discarded to give a mixture of a micronized fat cell and an adipose stem cell.

By undergoing such a separation step, a cell preparation including a finer fat cell and an adipose stem cell in a high concentration can be prepared. Here, a fine fat cell indicates a fat cell having a size such that it can pass through a needle of which gauge is 26 to 30, preferably 18 to 30. In the present step (3c″), a treatment to micronize the fat cell can be carried out at least once, preferably once to several times, more preferably once to twice. By micronizing the fat cell, the concentration of the adipose stem cell per unit volume of the cell preparation can be increased, and the effect of alleviating a disease of an interosseous joint or pain derived from muscle damage, the effect of regeneration of a tissue inside an interosseous joint and the effect of restoration of a muscle can be exhibited still more remarkably.

In addition, a step (4c″) of separating an adipose stem cell from at least a part of the mixture of a micronized fat cell and an adipose stem cell obtained in step (3c″) and mixing the obtained adipose stem cell with the concentrate obtained in step (2c) may be included. The method for separating an adipose stem cell is as described in regard to step (2b) of the method (b).

The fat cell obtained by separating the adipose stem cell in the present step (4c″) can be washed, and thereafter used as a cell preparation used in the treatment method of the present invention including a fat cell. Washing can be carried out by the method same as the above-mentioned washing treatment carried out for the adipose stem cell. Since a healthy, micronized fat cell is selected and concentrated, the fat cell obtained here exhibits a further remarkably excellent effect in alleviation of pain caused by a disease of an interosseous joint or muscle damage.

In the present method (c), by using the syringe described in JP 2007-533396, all of the steps can be carried out without contacting the cells with the open air. For example, a cannula or the like may be attached to the syringe described in JP 2007-533396, the syringe may be charged with the fat collected by liposuction, and the fat can be subjected to removal of impurities and a concentration treatment as it is to give a cell preparation. Thereafter, also upon administering the cell preparation, since the syringe can be connected to the pharmaceutical preparation injecting instrument of the present invention and administered to the affected part, a cell preparation having no problem such as contamination and high safety can be obtained. As an easy method, the present method (c) can be carried out using a commercially available kit, and a specific example of such a kit is LIPOMAX-SC (manufactured by Medikan International Inc.).

The cell preparation can be obtained by any of the above-mentioned methods, and it is preferable that the cell preparation is a cell preparation including a micronized fat cell preferably prepared via the method (b) or (c), more preferably via the method (c), further preferably via step (3″) or (4″) of the method (c). Since impurities have been removed and a micronized, healthy fat cell is included in a high concentration, the cell preparation including the micronized fat cell obtained via step (4″) of the method (c) exhibits a remarkable effect on treatment of a disease of an interosseous joint (especially alleviation of pain). The cell preparation including a micronized fat cell and an adipose stem cell obtained via step (3″) of the method (c) includes an adipose stem cell in a high concentration, in addition to a micronized, healthy fat cell. Such a cell preparation can exhibit a further remarkably excellent effect on treatment of a disease of an interosseous joint.

### EXAMPLES

### Preparation of a Cell Preparation

For preparation of the cell preparation, LIPOMAX-SC (manufactured by Medikan International Inc., Seoul, Korea) kit was used. In the preparation step of the present cell preparation, it was possible to carry out the step without contacting the cells with the open air at all. The specific method is as follows.

(i) Using a syringe (a syringe manufactured for LIPOMAX-SC: manufactured by Medikan International Inc., Seoul, Korea), liposuction was carried out on each subject. A syringe containing the collected fat was left standing still at room temperature (about 25° C.) for about 10 minutes to separate the fat into a cellular fraction and a liquid fraction, and the liquid fraction containing a tumescent fluid was discarded. The remaining cellular fraction was used in Test Example 2 as an adipose tissue. Thereafter, the syringe was subjected to centrifugation at 2,200×g for 8 minutes. By centrifugation, the cellular fraction was separated into three layers of the upper layer (free oil), the intermediate layer (a fat cell and an adipose stem cell) and the lower layer (a liquid fraction such as a cellular tissue fluid). Thereafter, only the intermediate layer was left in the syringe, and the upper and lower layers were discarded, thereby a concentrate of a fat cell and an adipose stem cell was obtained. The gelling treatment was carried out for the concentrate to micronize a relatively large fat cell. The gelling treatment was carried out once to three times. A drill (Filler-Geller cutter: manufactured by Medikan International, Inc.) was used for gelling to cut the fat cell for micronization, and the fat cell was further centrifuged at 2,200×g for 5 minutes, thereby a mixture including a micronized fat cell and an adipose stem cell was obtained. The mixture thus obtained was used in Test Examples 1 described below as a cell preparation including a micronized fat cell and an adipose stem cell.

Furthermore, since the fat cell and the adipose stem cell derived from each patient were used, the above-mentioned procedures were carried out for each patient.

### Test Example 1

The cell preparation including a micronized fat cell and an adipose stem cell prepared in the above-mentioned step (i) (5 to 10 mL, including about 750 thousand to 2 million adipose stem cells) was injected to the knee joint of 2 women having knee osteoarthritis. The site of the disease, the specific site of liposuction, the amount of the collected fat, the amount of injection of the cell preparation, and presence or absence of the procedure of stimulating the vicinity site of injection are shown in Table 1 below. Treatment for each subject, measurement results of the knee joint fissure and evaluation results on the basis of Kellgren and Lawrence classification which is an evaluation standard of an X-ray finding of knee osteoarthritis are summarized in Table 1 below.

The procedure of stimulating the vicinity site of injection was conducted by puncturing multiple (2 or 3 to about 10) vicinities of a diseased site by turning in all directions a cannula of which tip was obliquely cut. The stimulation of this procedure corresponds to that of the pharmaceutical preparation injecting instrument of the present invention.

TABLE 1

| | | Subject G | | Subject H | |
|---|---|---|---|---|---|
| Knee osteoarthritis | | Right | Left | Right | Left |
| Site from which fat was collected | | Lower abdomen | | Lower abdomen | |
| Amount of collected fat | | 76 mL | | 96 mL | |
| Presence or absence of | | + | − | − | + |
| procedure of stimulating | | | | | |
| site of injection by cannula | | | | | |
| Amount of injection of cell | | 8 mL | 6 mL | 6 mL | 8 mL |
| preparation | | | | | |
| Joint fissure | Before treatment | 0 | * | * | 2.4 |
| (mm) | After treatment | 1.8 | | | 4.4 |
| Kellgren and | Number of days | 7.5 weeks | | 8 weeks | |
| Lawrence | after treatment | | | | |
| classification | Before treatment | III | II | III | IV |
| (Grade) | After treatment | II | I | II | III |

* In the table, regarding knee osteoarthritis, left indicates that the subject has the disease in the left knee, and right indicates that the subject has the disease in the right knee.
* In the table, regarding presence or absence of procedure of stimulating site of injection by cannula, + indicates presence of stimulation, and − indicates absence of stimulation.
* In the table, regarding the size of joint fissure, − indicates that the size remained almost unchanged (under 0.5 mm).
* In the table, the number of days after treatment indicates the number of days elapsed from treatment until the Grade on the basis of Kellgren and Lawrence classification changed

In addition, the specific evaluation items and evaluation standard of Kellgren and Lawrence classification are as follows.

TABLE 2

| | Narrowing of | Osteophyte | | Deformation of |
|---|---|---|---|---|
| Grade | joint fissure | formation | Consolidation | outline of joint |
| I | Doubted | Possible | None | None |
| II | Possible | Clear | None | None |
| III | Clear | Moderate | Moderate | Possible |
| IV | Moderate | Profound | Profound | Clear |

According to the Table 1, it was shown that regeneration of the tissue in the knee joint was enhanced by injecting the cell preparation including a micronized fat cell and an adipose stem cell to the knee joint of the subject having knee osteoarthritis in both cases of the presence and the absence of stimulation. Regarding the detail of the treatment effect, improvement of pain of Subject G caused within the same day of treatment and the joint fissure was improved from 0 mm to 1.8 mm about 7.5 weeks after treatment by the presence of stimulation. On the other hand, by the absence of stimulation, improvement of pain caused within about 10 days and the joint fissure was not expanded substantially (under 0.5 mm) 7.5 weeks after treatment. Improvement of pain of Subject H caused and lasted from the same day of treatment and the joint fissure was improved from 2.4 mm to 4.4 mm 8 weeks after treatment by the presence of stimulation. On the other hand, by the absence of stimulation, improvement of pain caused within 8 days and the joint fissure was not expanded substantially (under 0.5 mm) 8 weeks after treatment. Without expecting limitative interpretation, these results show that early alleviation of arthralgia, the efficiency of engraftment of an adipose stem cell and the efficiency of regeneration of a tissue are augmented and then the treatment effect of knee osteoarthritis is improved because secretion of a cytokine or the like is promoted by giving appropriate stimulation to the vicinity of the site of injection and causes multiplier effect with the injected cell preparation.

The pharmaceutical preparation injecting instrument of the present invention achieves the above described effect since it can provide the stimulation of the target site in the Test Example 1 quickly and exactly.

### REFERENCE SIGNS LIST

1. Pharmaceutical preparation injecting instrument
10. Outer needle
12. Reference puncturing part
13. Attachment part
14. Inclined surface
15. Distal side opening
16. Proximal side opening
20. Inner needle
22. Periphery puncturing part
26. Tapered part

1. A pharmaceutical preparation injecting instrument comprising:
an outer needle formed in a straight tube shape having a distal side opening and a proximal side opening, the outer needle having a reference puncturing part on the distal side; and
an inner needle configured to be removably inserted in the outer needle, and having a periphery puncturing part on a distal side, wherein
the inner needle is configured to allow the periphery puncturing part to appear from, or disappear into, the distal side opening in accordance with an advancing or withdrawing operation on a base side,
in a state in which the inner needle is removed from the outer needle, the pharmaceutical preparation injecting instrument is configured to inject, from the distal side opening, a pharmaceutical preparation supplied from the proximal side opening, and
the outer needle is configured to guide the inner needle such that the periphery puncturing part projects to a side opposite to the reference puncturing part across a plane including an axial line.

2. The pharmaceutical preparation injecting instrument according to claim 1, wherein:
the reference puncturing part has a puncturing direction that is parallel to the axial line of the outer needle; and
the periphery puncturing part is configured to move away from the axial line as the periphery puncturing part advances from the distal side opening.

3. The pharmaceutical preparation injecting instrument according to claim 1, wherein the inner needle includes a holding part configured to regulate a length of projection from the distal side opening.

4. The pharmaceutical preparation injecting instrument according to claim 3, wherein:
the holding part is composed of a tapered part having a diameter that gradually decreases from the proximal side toward the distal side; and
the holding part is held by a circumference of the distal side opening.

5. The pharmaceutical preparation injecting instrument according to claim 1, wherein:
the distal side opening is formed in an inclined surface formed on the distal side of the outer needle so as to be off-center relative to a center of the outer needle; and
a tip of the reference puncturing part is located on a side opposite to a center of the distal side opening across the center of the outer needle as viewed from front.

6. The pharmaceutical preparation injecting instrument according to claim 1, wherein the outer needle includes, on the base side, a marked part that allows a position of the periphery puncturing part relative to the reference puncturing part to be recognized.

7. The pharmaceutical preparation injecting instrument according to claim 1, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

8. The pharmaceutical preparation injecting instrument according to claim 2, wherein the inner needle includes a holding part configured to regulate a length of projection from the distal side opening.

9. The pharmaceutical preparation injecting instrument according to claim 8, wherein:
the holding part is composed of a tapered part having a diameter that gradually decreases from the proximal side toward the distal side; and
the holding part is held by a circumference of the distal side opening.

10. The pharmaceutical preparation injecting instrument according to claim 2, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

11. The pharmaceutical preparation injecting instrument according to claim 3, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

12. The pharmaceutical preparation injecting instrument according to claim 4, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

13. The pharmaceutical preparation injecting instrument according to claim 5, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

14. The pharmaceutical preparation injecting instrument according to claim 6, wherein:
the outer needle is capable of insertion into a gap in a joint at which bones are joined to each other; and
in a state in which cartilage of a first bone is punctured by the reference puncturing part, the periphery puncturing part is capable of puncturing cartilage of a second bone.

15. The pharmaceutical preparation injecting instrument according to claim 2, wherein:
the distal side opening is formed in an inclined surface formed on the distal side of the outer needle so as to be off-center relative to a center of the outer needle; and
a tip of the reference puncturing part is located on a side opposite to a center of the distal side opening across the center of the outer needle as viewed from front.

16. The pharmaceutical preparation injecting instrument according to claim 3, wherein:
the distal side opening is formed in an inclined surface formed on the distal side of the outer needle so as to be off-center relative to a center of the outer needle; and
a tip of the reference puncturing part is located on a side opposite to a center of the distal side opening across the center of the outer needle as viewed from front.

17. The pharmaceutical preparation injecting instrument according to claim 4, wherein:
the distal side opening is formed in an inclined surface formed on the distal side of the outer needle so as to be off-center relative to a center of the outer needle; and
a tip of the reference puncturing part is located on a side opposite to a center of the distal side opening across the center of the outer needle as viewed from front.
